SEQUENCE LISTING (2 sequences)

SEQ ID NO: 1 - protein, 403 aa, Homo sapiens [sequence omitted]

SEQ ID NO: 2 - protein, 406 aa, Homo sapiens
MSGCPFLGNNFGYTFKKLPVEGSEEDKSQTGVNRASKGGLIYGNYLHLEKVLNAQELQSETKGNKIHDEHLFIITHQAYELWFKQILWELDSVREIFQNGHVRDERNMLKVVSRMHRVSVILKLLVQQFSILETMTALDFNDFREYLSPASGFQSLQFRLLENKIGVLQNMRVPYNRRHYRDNFKGEENELLLKSEQEKTLLELVEAWLERTPGLEPHGFNFWGKLEKNITRGLEEEFIRIQAKEESEEKEEQVAEFQKQKEVLLSLFDEKRHEHLLSKGERRLSYRALQGALMIYFYREEPRFQVPFQLLTSLMDIDSLMTKWRYNHVCMVHRMLGSKAGTGGSSGYHYLRSTVSDRYKVFVDLFNLSTYLIPRHWIPKMNPTIHKFLYTAEYCDSSYFSSDESD

(19) United States
(12) Patent Application Publication
(10) Pub. No.: US 2018/0252704 A1
(43) Pub. Date: Sep. 6, 2018

(54) SCREENING ASSAY TO IDENTIFY IDO1 AND/OR TDO MODULATORS

(71) Applicant: Hoffmann-La Roche Inc., Little Falls, NJ (US)

(72) Inventors: REMO HOCHSTRASSER, BASEL (CH); HAIYAN WANG, BASEL (CH)

(73) Assignee: Hoffmann-La Roche Inc., Little Falls, NJ (US)

(21) Appl. No.: 15/973,837

(22) Filed: May 8, 2018

Related U.S. Application Data
(63) Continuation of application No. PCT/EP2016/076765, filed on Nov. 7, 2016.

(30) Foreign Application Priority Data
Nov. 9, 2015 (EP) 15193667.1

(51) Int. Cl. G01N 33/50

(52) U.S. Cl. CPC G01N 33/5041; G01N 2333/90241

(57) ABSTRACT

The invention provides a cell based method for the identification of Indoleamine 2,3-dioxygenase 1 (IDO1) and/or tryptophan 2,3-dioxygenase (TDO) modulators.

### CROSS REFERENCE TO RELATED APPLICATIONS

This application is a continuation of International Application No. PCT/EP2016/076765 having an international filing date of Nov. 7, 2016, the entire contents of which are incorporated herein by reference, and which claims benefit under 35 U.S.C. § 119 to European Patent Application No. 15193667.1 filed Nov. 9, 2015.

### SEQUENCE LISTING

The instant application contains a Sequence Listing submitted via EFS-Web and hereby incorporated by reference in its entirety. Said ASCII copy, created on May 4, 2018 is named P33130US_Seq_List.txt, and is 7,353 bytes in size.

### FIELD OF THE INVENTION

The present invention relates to a cell based screening assay for the identification of Indoleamine 2,3-dioxygenase 1 (IDO1) and/or tryptophan 2,3-dioxygenase (TDO) specific modulators.

### BACKGROUND

Indoleamine 2,3-dioxygenase 1 (IDO1) and tryptophan 2,3-dioxygenase (TDO) are cytosolic, heme containing enzymes that catalyze the oxidative cleavage of tryptophan (Trp) to N-formylkynurenine (NFK), the first step in the kynurenine (Kyn) pathway. The depletion of Trp and formation of Kyn metabolites results in suppression of effector T-cell function and differentiation of regulatory T cells. Increased levels of IDO1 and/or TDO expression in tumor cells correlate with poor prognosis for survival in cancer. IDO has been clinically validated as a small-molecule drug target for cancer, while preclinical studies indicate that TDO may be a target for cancer immunotherapy (see reviews Lob et al, 2009; Platten et al, 2014 and references therein).

Pilotte et al, 2012) describe a low throughput mass spectrometry and high-performance liquid chromatography (HPLC) assay for testing of IDO1 and TDO inhibitors.

The present invention provides a high throughput compatible assay for the identification of IDO/TDO specific modulators.

### SUMMARY

The present invention provides a cell based method for the identification of Indoleamine 2,3-dioxygenase 1 (IDO1) and/or tryptophan 2,3-dioxygenase (TDO) modulators comprising:
a. providing cells recombinantly expressing IDO1 and/or TDO, wherein the IDO1 and/or TDO expression is inducible,
b. contacting the cells of step a) with a test compound and an IDO1 and/or TDO substrate,
c. contacting the mixture of step b) with a kynurenine sensor and
d. measuring the fluorescence readout of the mixture of step c), wherein an altered fluorescence readout in presence of the candidate compound compared to a blank is indicative for a modulator of IDO1 and/or TDO activity.

In a particular embodiment of the invention, the cells are HepG2 cells.

In a particular embodiment of the invention, the inducible IDO1 and/or TDO expression is due to the Tet-on system.

In a particular embodiment of the invention, the kynurenine sensor is 7-(diethylamino)-4-ethylsulfanyl-2-oxo-chromene-3-carbaldehyde (sensor1).

In a particular embodiment of the invention, the fluorescence is measured at Ex: 520-560 nm and Em: 580-680 nm.

In a particular embodiment of the invention, non-induced cells are used as blank.

In a particular embodiment of the invention, the IDO1 and/or TDO are human IDO1 and human TDO.

In a particular embodiment of the invention, the recombinantly expressing IDO1 and/or TDO cells are a stable cell line.

In a particular embodiment of the invention, in step d) the fluorescence readout of the supernatant of the mixture of step c) is measured.

In a particular embodiment of the invention, the method is performed in microtiter plates.

In a particular embodiment of the invention, a decreased fluorescence readout in presence of the candidate compound compared to a blank is indicative for an IDO1 and/or TDO inhibitor.

In a particular embodiment of the invention an increased fluorescence readout in presence of the candidate compound compared to a blank is indicative for an IDO1 and/or TDO activator.

In a particular embodiment of the invention, the IDO1 and/or TDO substrate is tryptophan.

The described assay is aimed to screen/profile and discover novel, highly potent IDO/TDO-selective and/or dual modulators, which are an immunotherapeutic agent that may help to break the immune tolerance within the tumor microenvironment, and prevent tumor escape from immune surveillance and destruction.

### BRIEF DESCRIPTION OF THE FIGURES

FIG. 1 shows an illustration of the inventive IDO1/TDO cell based screening assay.

FIG. 2A shows dose-dependent induction of IDO1 in HepG2-Tet-on IDO1 *29 cells. Immunoblotting of total cell lysates from HepG2-Tet-on-IDO1 *29 with Mouse anti-IDO1 mAb (*UM500091, Origene). Cells were treated with indicated concentrations of doxycycline for 24 h.

FIG. 2B shows dose-dependent induction of TDO in HepG2-Tet-on IDO1 *25 cells. Immunoblotting of total cell lysates from HepG2-Tet-on-TDO *25 with Mouse anti-TDO2 mAb (*TA504730, Origene). Cells were treated with indicated concentrations of doxycycline for 24 h.

FIG. 3A: shows HepG2-Tet-on-IDO1 *29 cells treated with indicated concentrations of doxycycline for 24 h. The assay with INCB024360 was performed as described in the Assay Methods. INCB024360=(Z)-N-(3-bromo-4-fluorophenyl)-N′-hydroxy-4-(2-(sulfamoylamino)ethylamino)-1,2,5-oxadiazole-3-carboximidamide. Example 1 in WO2010005958.

FIG. 3B. shows HepG2-Tet-on-TDO *25 cells treated with indicated concentrations of doxycycline for 24 h. The assay with INCB024360 was performed as described in the Assay Methods.

### DETAILED DESCRIPTION

A novel cell-based fluorescence assay was established to measure IDO1/TDO activity suitable for high-throughput screening of compound libraries for IDO and/or TDO modulators. This assay has opened up new areas of chemical space for discovery of inhibitors of two important drug targets. It relies on the quantification of the amount of kynurenine produced in the assay medium by Fluorescent Chemosensor (Sensor 1) (Klockow and Glass, 2013) and complements the standard low throughput mass spectrometry and high-performance liquid chromatography (HPLC) assay methods (Pilotte et al, 2012).

The term “IDO1” is used herein to refer to native sequence of Indoleamine 2,3-dioxygenase 1 from any animal, e.g. mammalian species, including humans, and IDO1 variants (which are further defined below).

“Native sequence IDO1” refers to a polypeptide having the same amino acid sequence as a IDO1 polypeptide occurring in nature regardless of its mode of preparation. A native sequence IDO1 may be isolated from nature, or prepared by recombinant and/or synthetic methods. The term “native sequence IDO1” specifically encompasses naturally occurring truncated or secreted forms, naturally occurring variant forms (e.g. alternatively spliced forms), and naturally occurring allelic variants of IDO1. The amino acid sequence of human IDO1 polypeptide is set forth in Seq. Id. No. 1.

The term “IDO1 variant” refers to amino acid sequence variants of a native sequence IDO1, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence IDOL

The term “TDO” is used herein to refer to native sequence of Indoleamine 2,3-dioxygenase from any animal, e.g. mammalian species, including humans, and TDO variants (which are further defined below).

“Native sequence TDO” refers to a polypeptide having the same amino acid sequence as a TDO polypeptide occurring in nature regardless of its mode of preparation. A native sequence TDO may be isolated from nature, or prepared by recombinant and/or synthetic methods. The term “native sequence TDO” specifically encompasses naturally occurring truncated or secreted forms, naturally occurring variant forms (e.g. alternatively spliced forms), and naturally occurring allelic variants of TDO. The amino acid sequence of human TDO polypeptide is set forth in Seq. Id. No. 2.

The term “TDO variant” refers to amino acid sequence variants of a native sequence TDO, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence TDO.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

Synthesis of kynurenine sensor 7-(diethylamino)-4-ethylsulfanyl-2-oxo-chromene-3-carbaldehyde (sensor 1).

The synthesis and development of Sensor 1 for the detection of Kynurenine has been described (Klockow and Glass, 2013).

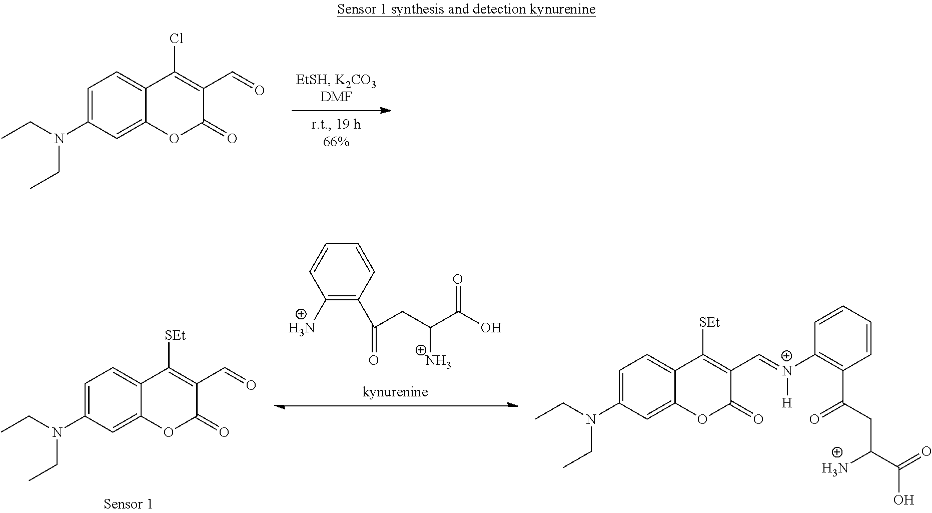

Generation of HepG2 stable cell lines allowing inducible expression of human IDO1 or human TDO2 in a doxycycline-dependent manner.

HepG2 cells were cultured in MEM (1X) +GlutaMax (*41090, Gibco®, Building 92-stock), 10% FBS (*16000-044, Gibco®). The first step of stable transfection was performed using plasmid pTet-On (*631018, Clontech) and Lipofectamine 2000 (*11668019, Life Technology) to establish cells expressing the reverse tetracycline-dependent transactivator. HepG2 cells in 10-cm dishes were transfected with 10 μg of pTet-On, followed by selection with 400 μg/ml G418 for 21 days, resistant colonies were isolated with cloning rings. Individual clones were tested for expression of reverse tetracycline-dependent transactivator by transient transfection of pTRE2-Luc (*S1496, Clontech), a reporter plasmid with the luciferase gene driven by the TetOn promoter. One clonal line that exhibited very high tetracycline-inducible luciferase activity and undetectable basal luciferase activity was chosen and used for a second round of transfection with a IDO1 or TDO2 expression plasmid. These plasmids were constructed by inserting either human IDO1 or TDO2 cDNAs (both purchased from the Origene) into pTRE2hyg expression vector (*631014, Clontech). After the secondary stable transfection and selection with 400 μg/ml hygromycin, individual resistant colonies were cloned and maintained in long-term culture with 400 μg/ml of G418 and 400 μg/ml of hygromycin. Clones were screened by Western blotting and two stable clones, assigned as HepG2-Tet-on-IDO1 *29 and HepG2-Tet-on-TDO *25, were selected based on high induction of IDO or TDO proteins after 24 h of cell culture with 1 μg/ml of doxycycline, but non-detectable under non-induced conditions.

INCB024360 compound=(Z)-N-(3 -bromo-4-fluorophenyl)-N′-hydroxy-4-(2-(sulfamoylamino)ethylamino)-1,2,5-oxadiazole-3-carboximidamide. INCB024630 is example 1 in WO2010005958.

Assay Methods: Cells were seeded 10,000 cells/well in the Cell Plate and incubated at 37° C. and 5% CO2 in the 95% humidified cell culture incubator. Cells were then induced with 1 μg/ml of doxycyline for 24 h to achieve full expression of IDO or TDO. Non-induced cells were used as 100% inhibition control. After washing 3 times with 60 μl of 1× HBSS (37° C.), cells were equilibrated with 60 μl of 1× HBSS in the cell culture incubator for 30 min. After an-other 3 times washing with 60 μl of 1× HBSS (37° C.), cells were treated with compounds for 10 min before adding the substrate, L-tryptophan, to a final concentration of 80 μM. The assay was carried out for 4 h at 37° C. and 5% CO2 in the cell culture incubator. The reaction was then stopped by transfer of 30 μl/well of supernatant into the Assay Plate. 10 μl/well of 30% (w/v) TCA were added and followed by application of Sensor 1 to a final concentration of 10 μM. After brief centrifugation, the fluorescence was measured at Ex: 546, Em: 586nm with a Paradigm (Molecular devices) plate reader.

| Step | Action | Parameter |
|---|---|---|
| Cell Culture | | |
| Day 0: Seed | | |
| cells | | |
| 1 | Seed 10,000 cells/well in Cell Plate | 40 μl |
| 2 | Incubate | 48 h, 37° C., 95% RH, 5% CO2 |
| Day 2: Induction | | |
| 3 | Add induction medium (5 μg/ml | 10 μl |
| | doxycycline) to CP | |
| 4 | Incubate | 24 h, 37° C., 95% RH, 5% CO₂ |
| Assay | | |
| Day 3: Cell | | |
| washing and | | |
| assay | | |
| 5 | Washing | 3X with 60 μl of 1X HBSS |
| 6 | Equilibrate | 30 min, 37° C., 95% RH, 5% CO₂ |
| 7 | Washing and Aspirate | 3X with 60 μl of 1X HBSS |
| 8 | Assay Buffer containing compounds | 40 μl |
| 9 | Incubate | 10 min, 37° C., 95% RH, 5% CO₂ |
| 10 | Add substrate (5X): 400 μM L- | 10 μl, 4 hours, 37° C., 95% RH, |
| | tryptophan in 1XHBSS | 5% CO₂ |
| 11 | Transfer supernatant to Assay Plate | 30 μl |
| 12 | Add 30% TCA | 10 μl |
| 13 | Add Sensor 1 (5X) (50 μM) | 10 μl |
| 14 | Centrifuge | 1,500 rpm, 5 min |
| 15 | Measure fluorescence | Ex: 546, Em: 586 nm |

Assay Materials:

Plates

Assay Plates Costar 384 well, all clear, NT, *3702

Cell Plates Costar 384 well, all clear, PDL coated, *356662

Cells and Buffer Solutions

| Reagent | | Stock | Final |
|---|---|---|---|
| (Brand) | Suppliers | Concentrations | Concentrations |
| HepG2-Tet-on- | Roche cell line | NA | 10000 cells/well |
| IDO1 *29 and | depository-Basel | | |
| HepG2-Tet-on- | | | |
| TDO *25 | | | |
| Doxycycline | Sigma D3447- | 1 mg/ml | 1 μg/ml |
| hydrochloride | 500MG | (freshly prepared) | |
| (SigmaAldrich) | 014M4043V | | |
| L-tryptophan | Sigma T0254- | 8 mM | 80 μM |
| | 100G | (store at −20° C.) | |
| Trichloroacetic | Sigma T6399- | 30% (w/v) | 6% (w/v) |
| acid | 500G | | |
| Sensor 1 | Synthesized at | 10 mM | 10 μM |
| | Roche | | |
| Washing and | 1X Hank's | NA | NA |
| Cell assay | Balanced | | |
| buffer | Salt Solution | | |
| | (1X HBSS) | | |
| | w. Mg2+ & Ca2+ | | |
| | (*14025-100, | | |
| | Life Technology) | | |

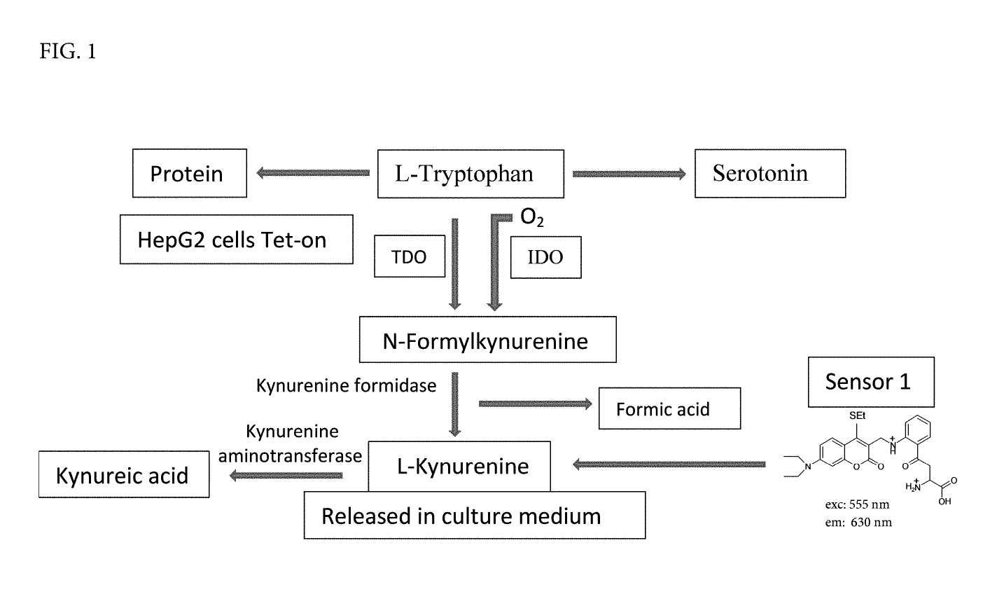

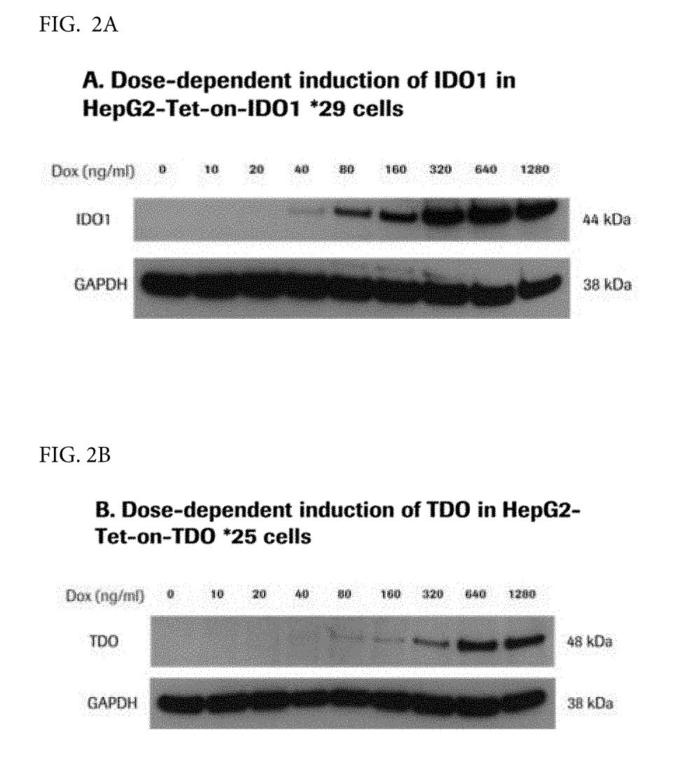

The present invention relates to a novel HTS-compatible cell based assay using inhouse established HepG2 stable cell lines allowing inducible expression of IDO or TDO. HepG2 cells do not express endogenous IDO1 nor TDO (Pilotte et al, 2012), therefore these enzymes could be induced in a tightly controlled doxycyline-dose-dependent manner, as demonstrated by Western blotting (FIG. 2A and 2B). The induced IDO1 or TDO converts tryptophan into N-formyl-kynurenine, which is subsequently metabolized to kynurenine by the abundant formamidase. Majority of the produced kynurenine is released into the culture medium, which is analyzed by the fluorescent “sensor I” probe (FIG. 1). The prior art biochemical IDOL but not the prior art biochemical TDO, assay is translational from enzyme assay to cell-based assay, confirming reports from the literature (Liu et al, 2010). Over 100-fold right-shift of IC50 from biochemical assay to cell-based assay is observed in TDO enzyme activity with reference compound, INCB024360. Therefore, the cell-based assay is more biologically relevant and reliable for determination of compound IC50.

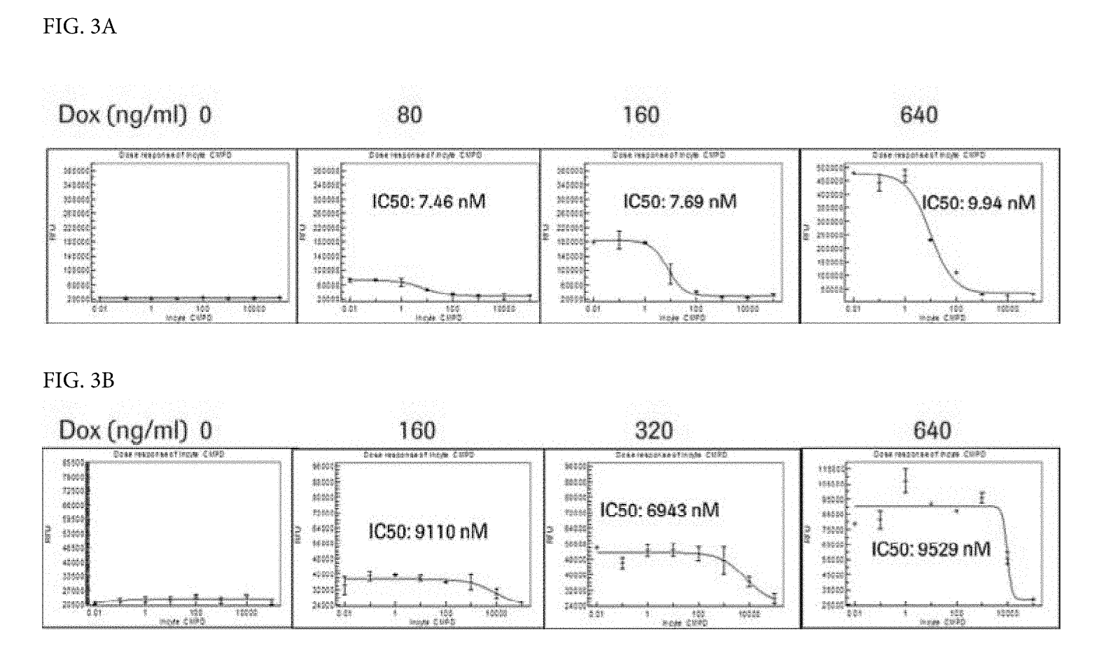

The inventive assay is not only a novel HTS-compatible cell-based assay for IDO or TDO, but also yields more reliable assay window comparing with literature (Liu et al., 2010). As shown in FIG. 3A and 3B, the assay window, though not IC50, is dependent on the expression levels of IDO and TDO in the cells. It is well known that the Tet-on inducible system allows much higher transgene expression comparing with endogenous or viral promoter mediated expression in most of mammalian cells (Gossen et al, 1995).

### References

Gossen M., Freundlieb S., Bender G., Muller G., Hillen W., Bujard H. (1995) Transcriptional activation by tetracyclines in mammalian cells, Science 268:1766-1769.

Hwu P., Du M. X., Lapointe R., Do M., Taylor M. W., Young H. A. (2000), Indoleamine 2,3-dioxygenase production by human dendritic cells results in the inhibition of T cell proliferation. The Journal of Immunology, 164:3596-3599

Klockow J. L. and Glass T. E. (2013), Development of a Fluorescent Chemosensor for the Detection of Kynurenine, Organic Letters, 15(2):235-237

Löb S., Königsrainer A., Rammensee H-G., Opelz G. and Terness P. (2009), Inhibitors of indoleamine-2,3-dioxygenase for cancer therapy: can we see the wood for the trees?, Nature Reviews/Cancer, 9:445-452.

Liu X., Shin N., Koblish H. K., Yang G., Wang Q., Wang K., Leffet L., Hansbury M. J., Thomas B., Rupar M., Waeltz P., Bowman K. J., Polam P., Sparks R. B., Yue E. W., Li Y., Wynn R., Fridman J. S., Burn T. C., Combs A. P., Newton R. C., Scherle P. A. (2010), Selective inhibition of IDO1 effectively regulates mediators of antitumor immunity, Blood, 115(17):3520-30.

Pilotte L., Larrieua P., Stroobanta V., Colaua D., Doluŝic′b E., Frédérickb R., De Plaena E., Uyttenhovea C., Woutersb J., Masereelb B., and Van den Eyndea B. J. (2012) Reversal of tumoral immune resistance by inhibition of tryptophan 2,3-dioxygenase, PNAS, 109(7): 2497-2502

Platten M., von Knebel Doeberitz N., Oezen I., Wick W. and Ochs K. (2014), Cancer immunotherapy by targeting IDO1/TDO and their downstream effectors, Frontiers in Immunology, 5 (673):1-7

What is claimed is:

1. A cell based method for the identification of Indoleamine 2,3-dioxygenase 1 (IDO1) and/or tryptophan 2,3-dioxygenase (TDO) modulators comprising:
a. providing cells recombinantly expressing IDO1 and/or TDO, wherein the IDO1 and/or TDO expression is inducible,
b. contacting the cells of step a) with a test compound and an IDO1 and/or TDO substrate,
c. contacting the mixture of step b) with a kynurenine sensor and
d. measuring the fluorescence readout of the mixture of step c), wherein an altered fluorescence readout in presence of the candidate compound compared to a blank is indicative for a modulator of IDO1 and/or TDO activity.

2. The cell based method of claim 1, wherein the cells are HepG2 cells.

3. The cell based method of claim 1 or 2, wherein inducible IDO1 and/or TDO expression is due to the Tet-on system.

4. The cell based method of claims 1 to 3, wherein the kynurenine sensor is 7-(diethylamino)-4-ethylsulfanyl-2-oxo-chromene-3-carbaldehyde (sensor1).

5. The cell based method of claim 4, wherein the fluorescence is measured at Excitation: 520-560 nm and Emission: 580-680 nm.

6. The cell based method of 1 to 5, wherein non-induced cells are used as blank.

7. The cell based method of claims 1 to 6, wherein the IDO1 and/or TDO are human IDO1 and human TDO.

8. The cell based method of claims 1 to 7, wherein the recombinantly expressing IDO1 and/or TDO cells are a stable cell line.

9. The cell based method of claims 1 to 8, wherein in step d) the fluorescence readout of the supernatant of the mixture of step c) is measured.

10. The cell based method of claims 1 to 9, wherein the method is performed in costar 384 well plates.

11. The cell based method of claims 1 to 10, wherein decreased fluorescence readout in presence of the candidate compound compared to a blank is indicative for an IDO1 and/or TDO inhibitor.

12. The cell based method of claims 1 to 10, wherein increased fluorescence readout in presence of the candidate compound compared to a blank is indicative for an IDO1 and/or TDO activator.

13. The cell based method of claims 1 to 12, wherein the IDO1 and/or TDO substrate is tryptophan.

14. The cell based method of claims 1 to 13, wherein the method is automated.
